(19) United States
(12) Patent Application Publication
(10) Pub. No.: US 2006/0029623 A1
(43) Pub. Date: Feb. 9, 2006

(54) Invert emulsions comprising at least one active agent sensitive to water and cosmetic/dermatological applications thereof

(75) Inventors: Fanny Astruc, Nice (FR); Sandrine Orsoni, Mandelieu (FR); Alain Brzokewicz, Antibes (FR)

Correspondence Address: BUCHANAN INGERSOLL PC;(INCLUDING BURNS, DOANE, SWECKER & MATHIS), POST OFFICE BOX 1404, ALEXANDRIA, VA, 22313-1404, US

(73) Assignee: GALDERMA RESEARCH & DEVELOPMENT, S.N.C., VALBONNE SOPHIA ANTIPOLIS (FR)

(21) Appl. No.: 11/196,301

(22) Filed: Aug. 4, 2005

Related U.S. Application Data
(63) Continuation of application No. PCT/EP04/04526, filed on Feb. 3, 2004.
(60) Provisional application No. 60/452,940, filed on Mar. 10, 2003.

(30) Foreign Application Priority Data
Feb. 5, 2003 (FR) 03/01349

(51) Int. Cl. A61K 31/59; A61K 9/00

(52) U.S. Cl. 424/400; 514/167

(57) ABSTRACT

Cosmetic/dermatological compositions useful for the treatment of a variety of dermatological conditions, including keratinization disorders, acne, eczema, psoriasis, etc., comprise an invert emulsion containing at least one active agent sensitive to the presence of water, notably a derivative of vitamin D, and solubilized in a glycolic or hydroglycolic dispersed hydrophilic phase, a lipophilic continuous phase and an emulsifier having an HLB ranging from 2 to 7; these invert emulsions exhibit good stability and avoid crystallization of the active agent and/or chemical degradation thereof.

### CROSS-REFERENCE TO PRIORITY/PCT/PROVISIONAL APPLICATIONS

This application claims priority under 35 U.S.C. § 119 of FR 03/01349, filed Feb. 5, 2003, and of provisional application Ser. No. 60/452,940, filed Mar. 10, 2003, and is a continuation of PCT/EP 2004/004526, filed Feb. 3, 2004 and designating the United States (published in the English language on Aug. 19, 2004 as WO 2004/069134 A3), each hereby expressly incorporated by reference and each assigned to the assignee hereof.

### BACKGROUND OF THE INVENTION

1. Technical Field of the Invention

The present invention relates to novel invert emulsion type compositions containing at least one active agent sensitive to the presence of water, and to applications thereof in cosmetics and dermatology.

2. Description of Background and/or Related and/or Prior Art

Human skin consists of two compartments, namely a deep compartment, the dermis, and a superficial compartment, the epidermis.

The dermis provides the epidermis with a solid support. It is also its feeder component. It consists mainly of fibroblasts and an extracellular matrix itself composed mainly of collagen, elastin and a substance, called ground substance. Leukocytes, mastocytes or tissue macrophages are also found therein. It also contains blood vessels and nerve fibers.

The epidermis is in contact with the external environment. Its role consists in protecting the body from dehydration and from external attacks, whether they are chemical, mechanical, physical or infectious.

The natural human epidermis is composed mainly of three types of cells, which are the keratinocytes, which are highly predominant, the melanocytes and the Langerhans' cells. Each of these cell types contributes by its specific functions to the essential role played in the body by the skin.

The cells constituting the epidermis are delimited by a lipid domain. The epidermal lipids are synthesized mainly in the living epidermis. They consist mainly of phospholipids, sphingolipids, cholesterol, free fatty acids, triglycerides, cholesterol esters and alkanes. During cell differentiation, the phospholipids, whose role consists in producing the fluid structure of the cell membranes of the living layers of the epidermis, are gradually replaced by a mixture mainly composed of fatty acids, cholesterol and sphingolipids, the essential constituents of the horny layer of the epidermis (stratum corneum).

The lipids of the intercorneocyte cement of the skin, and in particular the ceramides, are organized into lamellar bilayers or sheets and participate in the cohesion of the stratum corneum in order to maintain the integrity of the barrier and its protective, anti-penetration and anti-irritation role in particular.

Numerous active agents exhibit the difficulty of being very sparingly soluble in the commonly used cosmetic or pharmaceutical solvents, in particular water, and sensitive to an aqueous environment. This sensitivity to water can lead to chemical instability of the active agent and/or to crystallization of the active agent initially solubilized. This sensitivity to water therefore limits their formulation into cosmetic or dermatological compositions administered by the topical or oral route.

The phenomena of chemical degradation and/or crystallization of the active agent in the presence of water have as consequence a loss of efficacy and an uncertainty as regards the dose of active agent administered during its use, which runs counter to the desired objective. In addition, this degradation of the active agent and/or its crystallization can modify the overall stability of the compositions and their appearance.

The expression “active agent sensitive to the presence of water” according to the invention means active agents such as those chemically and/or physically unstable. For example, by chemically unstable, it will be understood that the active agent deteriorates in the composition. For example, by physically unstable, it will be understood that the active agent crystallizes or precipitates in the composition.

The galenic form currently most commonly used in dermatology is the oil-in-water emulsion in which the active agent is preferably solubilized in the lipophilic phase. However, this solution remains scarcely satisfactory because to respond to an objective of an active agent concentration having a quantifiable therapeutic efficacy would require very high concentrations of solvent oils, leading to products which are undoubtedly not very pleasant to use, due to their sticky feel, and which are physically unstable while retaining a limited active agent concentration.

Moreover, the solubilization of the active agent in the internal phase of the emulsion limits its release, the external aqueous or hydroglycolic phase constituting, for the active agent, a physical barrier to its release and its diffusion towards the skin layers.

Another option is to solubilize the active agent in the external hydrophilic phase of the emulsion, within the limit of its solubility in the aqueous or hydroglycolic media. However, this solution does not make it possible to solve the problems of chemical stability encountered, because the water activity of the emulsion remains very high.

The replacement of all or part of the aqueous phase with one or more glycols would lead to formulations which are cosmetically not very acceptable. It is indeed known to those skilled in the art that above 20% glycol, the formulation is cosmetically not very acceptable by virtue of its sticky feel, and its physical stability would not be guaranteed.

The production of an invert emulsion (the expression “invert emulsion” means an emulsion of the hydrophilic phase dispersed in lipophilic phase type) as an alternative was not obvious to those skilled in the art, given the known difficulties of formulating active agents exhibiting problems of chemical instability and/or crystallization from water.

The use of hydrophilic solubilizing agents such as propylene glycol was also not apparent to those skilled in the art, given that the high concentrations necessary were not favorable to a good physical stability of the formula and to an acceptable cosmetic feel.

The obtaining of good tolerance with solubilizing agents such as propylene glycol was also not obvious because phenomena of skin intolerance had been shown in humans, for example in healthy humans (Motoyoshi et al., Cosmet. and toiletries, 99, 83-89, 1984).

Need therefore continues to exist for a composition which makes it possible to respond to one or more of the following aspects: to have good stability of the formula to cold and to heat, in particular as regards maintaining the size of the globules and the absence of phase separation, to have good resistance of the active agent to the phenomena of oxidation, to allow good chemical stability of the active agent and good availability thereof for the skin, to exhibit good skin tolerance. It would also be useful to provide a composition allowing a high dispersed volume fraction. It is moreover useful for the preparation of such compositions to benefit from an advantageous mode of preparation.

### SUMMARY OF THE INVENTION

A formulation of the glycol-in-oil type has now surprisingly been developed which makes it possible to dispense with the various problems linked to the abovementioned aspects while making it possible in particular to have good physical stability of the composition as such, but also to allow good chemical stability and availability of the active agent which it contains. The compositions according to the invention also provide the advantage of exhibiting good skin tolerance and allowing a high dispersed volume fraction.

Thus, the present invention therefore features compositions containing at least one active agent sensitive to the presence of water, comprising invert emulsions containing a glycolic or hydroglycolic dispersed hydrophilic phase, a lipophilic continuous phase and an emulsifier having an HLB of from 2 to 7.

### DETAILED DESCRIPTION OF BEST MODE AND SPECIFIC/PREFERRED EMBODIMENTS OF THE INVENTION

The term HLB means the Hydrophilic/Lipophilic Balance (HLB), which corresponds to the balance from the size and strength of the hydrophilic group and the size and strength of the lipophilic group of the emulsifier.

This invention also makes it possible to obtain good release/penetration of the active agent at the level of the various skin layers, leading to good availability of the active agent in the skin, the said active agent being used in solubilized form.

The expression “solubilized form” means a dispersion in the molecular state in a liquid, no crystallization of the active agent being visible with the naked eye or even under a cross-polarization optical microscope.

The formulation of the solubilized active agents in a glycolic or hydroglycolic phase into an invert emulsion according to the invention thus makes it possible, surprisingly, to dispense with the problems of chemical stability and crystallization commonly encountered in formulations with the type of active agent used according to the invention.

The present invention therefore features preparing invert emulsions, containing a glycolic or hydroglycolic hydrophilic phase, which are perfectly stable (size of the globules and viscosity), even at a high dispersed volume fraction, showing no chemical degradation and/or crystallization of the active agent.

The present invention also features the preparation of invert emulsions containing an active agent solubilized in the lipophilic phase of the emulsion, and exhibiting good physicochemical stability, and no crystallization of the active agent.

The expression active agent sensitive to the presence of water and to be formulated into a composition according to the invention is understood to mean active agents such as those of the family of steroids, prostaglandins, carotenoids, synthetic retinoids, derivatives of vitamin D, tetracyclines, minoxidil or its analogues.

The preferred active agents according to the invention are the derivatives of vitamin D.

The expression “derivatives of vitamin D” means compounds which exhibit biological properties similar to those of vitamin D, in particular the properties of transactivation of the vitamin D response elements (VDRE), such as an agonist or antagonist activity towards receptors for vitamin D or its derivatives. The expression D vitamins or their derivatives is understood to mean, for example, the derivatives of vitamin D₂ or D₃ and in particular 1,25-dihydroxyvitamin D₃ (calcitriol).

By way of nonlimiting examples of preferred active compounds according to the invention, there may be mentioned those described and claimed by the applicant in WO 00/26167 and WO 00/65293, and more particularly (4E,6E)-7-[3-(3,4-bishydroxymethylbenzyloxy)phenyl]-3-ethylnona-4,6-dien-3-ol and {4-[6-Ethyl-4′-(1-ethyl-1-hydroxy-propyl)-2′-propyl-biphenyl-3-yloxymethyl]-2-hydroxymethyl-phenyl}-methanol.

The compositions according to the invention are preferably suitable for topical application onto the skin, the superficial body growths and/or the mucous membranes. Same generally contain a physiologically acceptable medium and a sufficient quantity of active compound to obtain the desired effect. The proportion by weight of active agent, relative to the total weight of the composition, may thus be from 0.001% to 20% (weight/weight), for example from 0.1 to 20%, in particular from 0.2 to 10%, especially from 0.2 to 4%, for example from 0.2 to 2%.

The glycols according to the present invention may be defined as alkylene or polyalkylene glycols. By way of nonlimiting examples, there may be mentioned alkylene and polyalkylene glycols (C1 to C6) such as ethylene glycol, polyethylene glycol (2 to 20 monomers), propylene glycol, dipropylene glycol, butylene glycol, pentylene glycol and hexylene glycol. They may be oxyethylenated or otherwise (2 to 50 EO). Those preferred according to the invention are hexylene glycol, propylene glycol and dipropylene glycol, and polyethylene glycol 400 (PEG 400).

The glycols which can be used according to the invention will advantageously have, as solubility parameter, a δp of less than 10, it being understood that the 3 Hansen solubility parameters—δd, δp and δh—characterize, for a given constituent, the energies corresponding respectively to the dispersive, polar and hydrogen bond type interactions existing from the molecules of this constituent, δp characterizing more particularly the Debye forces of interaction from dipoles and being a function of the number of oxygen atoms in the formula of the given constituent (S. paint Technology, 30, 195, 1967, “The three dimensional solubility parameter—Key to paint component affinities”).

As lipophilic compounds which can be used to constitute the continuous fatty phase of the emulsions according to the invention, there may be mentioned mineral oils (paraffin oil), oils of plant origin (avocado oil, soya-bean oil), oils of animal origin (lanolin), synthetic oils (perhydrosqualene), silicone oils (cyclomethicone, dimethicone) and fluorinated oils (perfluoropolyethers). It is also possible to use fatty alcohols such as cetyl alcohol, Guerbet alcohols, in particular octyidodecanol known under the name Eutanol G, fatty acids, waxes, gums and in particular silicone gums.

The fatty phase may also consist of linear or branched mono-, di- or triesters of synthetic origin, in particular isopropyl myristate or palmitate, or caprylic/capric triglyceride (Miglyol 812).

Preferably, nonoxidizable compounds are used to constitute the oils of the continuous lipophilic phase, which are preferably selected from those of the silicone type, those of the ester type or those of the mineral type.

The compounds entering into the composition of the lipophilic phase of the emulsion will have as Hansen solubility parameter a δp of less than 5, and for example of from 0 to 2.

Moreover, in order to avoid any crystallization of the active ingredient, the overall solubility parameter for the lipophilic phase—δtₜ=√δd+δp+δh—will have a value of less than 20, for example of from 10 to 20, and preferably of from 12 to 18.

The volume fraction of the dispersed hydrophilic phase in the emulsion according to the invention ranges from 10 to 90% relative to the total volume of the emulsion. It may be exclusively glycolic or hydroglycolic. The volume proportion of glycols (relative to the total volume of the dispersed phase) is from 10 to 100%, for example from 30 to 100%, in particular from 60 to 100%, and preferably from 80 to 100%.

For a cosmetic application, from 30 to 50% of glycols will be preferably used (a proportion relative to the total volume of the dispersed phase).

It is also possible to characterize a preferred embodiment of the invention with reference to the water activity (a_{w}) of the hydrophilic phase in the composition according to the invention.

The invention thus also features, specifically, compositions as defined above, characterized in that the water activity a_{w} of the hydrophilic phase is less than 0.85.

The water activity a_{w} of a medium containing water is the ratio of the vapor pressure of water in the product “P_{H2O} product” and the vapor pressure of pure water “P_{H2O} pure” at the same temperature. It may also be expressed as the ratio of the number of water molecules “N_{H2O}” to the total number of molecules “N_{H2O}+N_{dissolved substances}”, which takes into account those of the dissolved substances “N_{dissolved substances}”

It is given by the following formulae:

$${a}_{w} = \frac{{P}_{H2O} product}{{P}_{H2O} pure} = \frac{{N}_{H2O}}{{N}_{H2O} + {N}_{dissoved substaces}}$$

It is possible to use various methods for measuring the water activity a_{w}. The most common is the manometric method by which the vapor pressure is measured directly.

Conventionally, a cosmetic or dermatological composition has a water activity of around 0.95 to 0.99. A water activity of less than 0.85 represents a substantial reduction.

Emulsifiers (or surfactants) are natural or synthetic substances consisting of a hydrophilic or polar part and a lipophilic or apolar part. They are amphiphilic molecules since they have a double polarity. Emulsifiers are characterized by their HLB; if the HLB is high, the hydrophilic part is predominant, if the HLB is low, the lipophilic part predominates.

Among these emulsifiers are preferably included polymeric emulsifiers which are characterized by a high molar mass and a nonlinear structure which allows greater anchorage at the water/oil interface than that obtained with the monomer-type emulsifiers.

The emulsifiers which it is possible to use according to the invention, alone or as a mixture, are those which make it possible to make invert emulsions having an HLB of less than 7.

In general, the preferred emulsifiers are the silicone emulsifiers, of the organopolysiloxane type, such as:

E1) polyalkyl methicone copolyols (oxyalkylenated, optionally crosslinked polyalkyl methylsiloxanes) containing:
linear or branched, saturated or unsaturated, C6 to C20 alkyl chains
a polyoxyethylenated unit of 1 to 50 EO (ethylene oxide) and/or
a polyoxypropylenated unit of 1 to 50 PO (propylene oxide)

E2) oxyalkylenated polyalkyl dimethyl methylsiloxanes containing:
linear or branched, saturated or unsaturated, C6 to C20 alkyl chains
a polyoxyethylenated unit of 1 to 50 EO and/or
a polyoxypropylenated unit of 1 to 50 PO

The organopolysiloxanes of the composition of the invention contain in particular one or more oxyalkylenated and in particular oxyethylenated (EO) groups, for example from 1 to 40 oxyalkylenated units, preferably from 1 to 20, even better from 10 to 20, more preferably from 12 to 20 and better still from 12 to 18 oxyalkylenated units, which can form polyoxyalkylenated and in particular polyoxyethylenated chains. These groups may be pendant or at the chain end. The silicon atoms carrying these groups are advantageously about 1 to 10 and even better 1 to 6 in number. The silicone structure forming the polymeric backbone of the organopolysiloxane containing (an) oxyalkylenated group(s) is advantageously a polydimethylsiloxane (PDMS) structure of which some of the methyl groups are optionally substituted with C2 to C30, and preferably C8 to C24, and even better from C10 to C20, alkyl groups or phenyl groups, at the chain end or pendant.

Advantageously, there will therefore be used as E1 or E2 type emulsifiers silicone emulsifiers such as alkyl dimethicone copolyols such as Abil EM-90, or the dimethicone copolyol and cyclomethicone mixture, sold by Dow Corning under the name 3225C Formulation Aid, lauryl methicone copolyol sold under the name Emulsifier 10 by Dow Corning, or mixtures based on a silicone polymer such as cetyl dimethicone copolyol with polyglyceryl-4 isostearate and hexyl laurate sold under the name Abil WE09 by Goldschmidt, Abil EM97 from Goldschmidt (dimethicone copolyol & cyclomethicone), Wacker SPG 128 VP from Wacker (cyclomethicone and octyl dimethicone methoxy glycosyl), or alternatively Silwax WD-IS (dimethicone copolyol isostearate).

E3) Mono- or polyalkyl ester siloxanes, for example Silwax S from Lambent (dimethiconol stearate),

E4) alkoxylated carboxylic acid esters such as polyhydroxylated alkyl esters of PEG, for example Arlacel P 135 from Uniqema (PEG-30 dipolyhydroxystearate).

There will be preferably used emulsifiers having an HLB of from 2 to 7, preferably a silicone W/O emulsifier having an HLB of from 2 to 7, preferably a polymeric silicone W/O emulsifier having an HLB of from 2 to 7.

The invert emulsions of the invention may be, as a variant, produced and stabilized with emulsifiers or with the following combinations with an emulsifying character:
1) the combination of an oxyalkylenated crosslinked elastomeric organopolysiloxane and a crosslinked and at least partially neutralized poly(2-acrylamido-2-methylpropanesulfonic acid) polymer.

The compositions according to the invention will contain in particular, expressed as a percentage by weight, from 0.5 to 8% of emulsifier, for example from 0.5 to 5%, preferably from 3 to 5%, relative to the total weight of the composition.

Moreover, advantageously, to improve the stability of the dispersion, it is possible to combine the principal emulsifiers described above with one or more coemulsifiers having an HLB of greater than 6. The (coemulsifier/emulsifier) ratio will be advantageously less than 1.5, and preferably less than 0.75.

By way of example, there may be mentioned:
polyoxyethylenated or nonpolyoxyethylenated alkyl or polyalkyl esters of sorbitan with from 1 and 5 branched or unbranched, saturated or unsaturated C10 to C20 alkyl chains, and with from 0 to 40 EO (for example sorbitan monolaurate 20 EO or sorbitan monooleate 20 EO (Tween 80 from Uniqema));
polyoxyethylenated alkyl or polyalkyl ethers or esters with from 1 and 5 branched or unbranched, saturated or unsaturated C10 to C20 alkyl chains and with 0 to 40 EO (ceteareth-20 (Eumulgin B2 from Cognis), or steareth (Brij 78) 20 EO);
ethoxylated and esterified alkyl or polyalkyl mono- or polyglucosides with from 1 and 5 branched or unbranched, saturated or unsaturated C6 to C20 alkyl chains and from 1 to 10 glucose units (for example PEG 20 methyl glucose sesquistearate (Glucamate SSE-20 from Amerchol));
polyglycerol alkyl or polyalkyl esters or ethers with from 1 and 5 branched or unbranched, saturated or unsaturated C10 to C20 alkyl chains and from 1 to 8 glycerol units (for example polyglyceryl4 isostearate or PEG-8 stearate (Myrj 45)).

Finally, it is possible to advantageously add to the dispersed phase from 0 to 10% by weight, relative to the total weight of the formulation, of a cosolvent for the active agent having an evaporation temperature of less than 100° C., preferably linear or branched C1 to C4 alcohols, such as ethanol and isopropanol.

Advantageously, the preparation of the emulsions according to the invention was found to require only little mechanical or thermal energy compared with the preparations of other invert emulsions already known.

In a known manner, the compositions of the invention may also contain the adjuvants customarily used in the cosmetic and dermatological fields, such as hydrophilic or lipophilic gelling agents, humectants such as glycerine and sorbitol, fatty phase thickeners, preservatives, antioxidants, electrolytes, solvents, perfumes, fillers, screening agents, pigments, odor absorbers, coloring matter and metal-chelating agents. The quantities of these various adjuvants are those conventionally used in the fields considered, and are for example from 0.01 to 20% of the total weight of the composition. These adjuvants, depending on their nature, may be introduced into the lipophilic phase or into the hydrophilic phase. These adjuvants, and their concentrations, should be such that they do not adversely affect the cosmetic and/or dermatological properties of the composition according to the invention.

As hydrophilic gelling agents, there may be mentioned in particular carboxyvinyl polymers (carbomer), acrylic copolymers such as acrylate/alkyl acrylate copolymers, polyacrylamides, polysaccharides, natural gums and clays, and, as lipophilic gelling agents, there may be mentioned modified clays such as bentones, metal salts of fatty acids and hydrophobic silica.

The compositions according to the invention have a cosmetically acceptable feel, good skin tolerance, good physical stability, that is to say absence of phase separation and retention of the size of the globules in the cold (at 4° C.) and in heat (45° C.) over a long period, for example over 2 months, with a stable viscosity over this period. The compositions according to the invention also make it possible to confer on the active agent good chemical stability and to avoid its crystallization over time.

In particular, the present invention features cosmetic or dermatological compositions for topical application to the skin, the superficial body growths and/or the mucous membranes, in the form of an invert emulsion containing a dispersed glycolic or hydroglycolic hydrophilic phase and a lipophilic continuous phase, comprising, in a physiologically acceptable medium (that is to say compatible with topical application to the skin, the superficial body growths and/or the mucous membranes), expressed as a percentage by weight:
from 0.001 to 5% of at least one active agent sensitive to the presence of water and more particularly of a derivative of vitamin D,
from 30 to 100% of glycols,
from 0.5 to 8% of emulsifier having an HLB of from 2 to 7,
from 0% to 5% of coemulsifier having an HLB of greater than 6,
from 0 to 50% of water, for example from 0 to 20% of water.

In a particular embodiment of the invention, the dispersed hydrophilic phase has a water activity of less than 0.85.

The present invention also features the administration (regime or regimen) of the novel invert emulsions as described above in cosmetics and in dermatology.

By virtue of the activity of the preferred compounds used in the composition, the composition according to the invention finds application in the prevention and/or treatment of the following pathologies:
1) for treating dermatological conditions or afflictions linked to a keratinocyte or sebocyte differentiation or proliferation disorder, in particular for treating acne vulgaris, comedo-type acne, polymorphic acne, acne rosacea, nodulocystic acne, acne conglobata, senile acne, secondary acne such as solar acne, acne medicamentosa or occupational acne;
2) for treating keratinization disorders, in particular ichthyosis, ichthyosiform states, Darier's disease, keratosis palmaris et plantaris, leukoplakia and leukoplakia-like states, skin or mucosal (buccal) lichen;
3) for treating other dermatological conditions linked to a keratinization disorder with an inflammatory and/or immunoallergic component, and in particular all forms of psoriasis, whether cutaneous, mucosal or ungual, and even arthropathic psoriasis, or skin atopy, such as eczema, or respiratory atopy or gingival hypertrophy;
4) for treating certain inflammatory skin conditions not exhibiting keratinization disorder, such as atopic eczema and contact allergies;
5) for treating all dermal or epidermal proliferations, whether benign or malignant, whether of viral origin or otherwise, such as verruca vulgaris, verruca plana and epidermodysplasia verruciformis, oral or florid papillomatosis and proliferations which may be induced by ultraviolet radiation, in particular in the case of baso- and spinocellular epithelioma;
6) for treating other dermatological disorders such as bullous dermatosis and collagen diseases;
7) for preventing or treating the signs of skin aging, whether photoinduced or chronological, or for reducing actinic pigmentations and keratoses, or any skin pathologies associated with chronological or actinic aging;
8) for preventing or treating cicatrization disorders or for preventing or repairing stretch marks;
9) for combating sebaceous function disorders such as acne hyperseborrhoea or ordinary seborrhoea or seborrhoeic eczema;
10) for treating certain ophthalmological disorders, in particular corneopathies;
11) for the treatment or prevention of the cancerous or precancerous states of skin or nonskin cancers exhibiting or capable of being induced to exhibit vitamin D receptors, such as, but without limitation, breast cancer, leukemia, myelodysplastic syndromes and lymphomas, carcinomas of the cells of the malpighian epithelium and gastrointestinal cancers, melanomas, and osteosarcoma;
12) for the treatment of inflammatory conditions such as arthritis or rheumatoid arthritis;
13) for the treatment of any condition of viral origin at the cutaneous or general level;
14) for the prevention or treatment of alopecia of various origins, in particular alopecia due to chemotherapy or to radiation;
15) for the treatment of dermatological or general conditions with an immunological component;
16) for the treatment of immunological conditions, such as autoimmune diseases (such as, but without limitation, type 1 diabetes mellitus, multiple sclerosis, lupus and lupus-type conditions, asthma, glomerulonephritis, etc.), selective dysfunction of the immune system (for example AIDS) and the prevention of immune rejection, such as graft rejections (for example the kidney, the heart, the bone marrow, the liver, pancreatic islets or the whole pancreas, the skin, etc.) or the prevention of graft-versus-host disease;
17) for the treatment of endocrine conditions which can be treated with vitamin D analogues such as those advantageously modulating hormone secretion, for example, by increasing the secretion of insulin or selectively suppressing the secretion of the parathyroid hormone (for example in chronic renal insufficiency and secondary hyperparathyroidism);
18) for the treatment of conditions characterized by an abnormal management of intracellular calcium; and
19) for the treatment or prevention of vitamin D deficiency and other conditions related to mineral homeostasis in the plasma and the bones, such as rickets, osteomalacia, osteoporosis, in particular in the case of menopausal women, renal osteodystrophy, and parathyroid function disorders.

The compositions according to the invention also find application in the cosmetic field, in particular in body and hair hygiene and in particular for the treatment of skins with a tendency to develop acne, for hair regrowth or for slowing its loss, for combating the greasy appearance of the skin or the hair, in protecting against the harmful effects of the sun or in the treatment of dry skins, for preventing and/or treating photoinduced or chronological skin aging.

This invention also features the pharmaceutical preparations and the medicaments obtained from the compositions according to the invention.

The present invention therefore features compositions containing at least one active agent sensitive to the presence of water, which is not DHEA and/or its chemical and/or biological precursors or derivatives, said compositions being invert emulsions containing a glycolic or hydroglycolic dispersed hydrophilic phase, a lipophilic continuous phase and an emulsifier having an HLB of from 2 to 7.

The compositions according to the invention are characterized in that the active agent is selected from the group consisting of a synthetic retinoid, a derivative of vitamin D and a derivative of minoxidil, and preferably a derivative of vitamin D, (4E,6E)-7-[3-(3,4-bishydroxymethylbenzyloxy)phenyl]-3-ethylnona-4,6-dien-3-ol and {4-[6-Ethyl-4′-(1-ethyl-1-hydroxy-propyl)-2′-propyl-biphenyl-3-yloxymethyl]-2-hydroxymethyl-phenyl}-methanol.

The compositions according to the invention are characterized in that they also contain one or more active agents selected from isoflavonoids, metalloproteinase inhibitors, carotenoids, anti-glycation compounds, NO-synthase inhibitors, vitamins, desquamating agents, compounds increasing the synthesis of glycosaminoglycans, anti-irritant compounds, compounds reducing irritation of neurogenic origin, muscle-relaxing compounds and depigmenting agents.

The compositions according to the invention contain from 0.001 to 20% by weight of the active agent sensitive to the presence of water, relative to the total weight of the composition, and more particularly the composition contains from 0.01 to 4% by weight of derivatives of vitamin D, relative to the total weight of the composition.

The compositions according to the invention are characterized in that the emulsifier is a silicone emulsifier, selected from lauryl methicone copolyol, cetyl dimethicone copolyol, a mixture of dimethicone copolyol and cyclomethicone or a mixture of cetyl dimethicone copolyol with polyglyceryl-4 isostearate and hexyl laurate.

The compositions according to the invention are characterized in that they also contain a coemulsifier having an HLB of greater than 6, which is preferably ceteareth-20.

In the compositions according to the invention, the proportion by volume of glycol, relative to the total volume of the dispersed phase, is from 10 to 100%, the glycol being preferably selected from propylene glycol, hexylene glycol, dipropylene glycol and PEG 400.

The compositions according to the invention are characterized in that the water activity of the dispersed hydrophilic phase is less than 0.85.

The present invention also features cosmetic or dermatological compositions for topical application to the skin, the superficial body growths and/or the mucous membranes, in the form of an invert emulsion containing a dispersed glycolic or hydroglycolic hydrophilic phase and a lipophilic continuous phase, comprising, in a physiologically acceptable medium, expressed as a percentage by weight:
from 0.001 to 5% of an active agent sensitive to the presence of water, other than DHEA and/or its chemical and/or biological precursors or derivatives,
from 30 to 100% of glycols,
from 0.5 to 8% of an emulsifier having an HLB of from 2 to 7,
from 0% to 5% of a coemulsifier having an HLB of greater than 6,
from 0 to 50% of water, for example from 0 to 20% of water.

The present invention features the administration of the compositions according to the invention for the prevention or treatment:
of dermatological conditions linked to a keratinocyte or sebocyte differentiation or proliferation disorder;
of keratinization disorders;
of dermatological conditions linked to a keratinization disorder with an inflammatory and/or immunoallergic component;
of inflammatory skin conditions not exhibiting a keratinization disorder;
of dermal or epidermal proliferation;
of dermatological disorders such as bullous dermatosis and collagen diseases;
of the signs of skin aging, whether photoinduced or chronological, or for reducing actinic pigmentations and keratoses, or any skin pathologies associated with chronological or actinic aging;
of cicatrization disorders and stretch marks;
of sebaceous function disorders such as acne hyperseborrhoea or ordinary seborrhoea or seborrhoeic eczema;
of dermatological conditions with an immunological component.

More particularly, this invention features the administration of a subject composition for the prevention or treatment of acne vulgaris, comedo-type acne, polymorphic acne, acne rosacea, nodulocystic acne, acne conglobata, senile acne, secondary acne such as solar acne, acne medicamentosa or occupational acne, ichthyosis, ichthyosiform states, Darier's disease, keratosis palmaris et plantaris, leukoplakia and leukoplakia-like states, skin or mucosal (buccal) lichen, various forms of psoriasis, whether cutaneous, mucosal or ungual, arthropathic psoriasis, and skin atopy, such as eczema, or respiratory atopy or gingival hypertrophy.

This invention also features the subject compositions as medicaments for the treatment or prevention of the pathologies noted above.

In order to further illustrate the present invention and the advantages thereof, the following specific examples are given, it being understood that same are intended only as illustrative and in nowise limitative. In said examples to follow, all parts and percentages are given by weight, unless otherwise indicated.

### EXAMPLE 1

### Solubility of the Vitamin D Derivative in Various Excipients

In the table below are the solubility results for (4E,6E)-7-[3-(3,4-bishydroxymethylbenzyloxy)phenyl]-3-ethylnona4,6-dien-3-ol, a vitamin D analogue, in various excipients:

| Solution | Concentration in % (w/w) |
|---|---|
| Propylene glycol | 6.7 |
| Ethanol Rectapur | 23.3 |
| Transcutol | 27.0 |
| Eutanol G | 0.5 |
| Miglyol 812 | 0.4 |
| Tegosoft TN | 0.3 |
| Crodamol IPP | 0.2 |
| PEG 400 | 17.8 |
| pH 4 | <0.05 |
| pH 7 | <0.05 |
| Silicone DC 200 | <0.05 |
| Primol 352 | <0.05 |
| Marcol 172 | <0.05 |
| Glycerine | <0.05 |
| Cetiol SN | <0.1 |

These maximum solubilities of (4E,6E)-7-[3-(3,4-bishydroxymethylbenzyloxy)phenyl]-3-ethylnona-4,6-dien-3-ol, a vitamin D analogue, were measured after stirring for 12 h with a magnetic stirrer bar, at room temperature, with an excess of active ingredient in the excipient to be analyzed. The suspension is then filtered (1.2 μm) and then the filtrate is assayed by HPLC.

### EXAMPLES 2 TO 10

### Methods of Preparation

In the examples below (Examples 2 to 10), the proportions of the various constituents are expressed as percentages by weight, unless otherwise stated. The active compounds used are the following:
Compound 1: (4E,6E)-7-[3-(3,4-bishydroxymethyl-benzyloxy)phenyl]-3-ethylnona-4,6-dien-3-ol;
Compound 2: {5-[6,2′-diethyl-4′-(1-ethyl-1-hydroxy-propyl)biphenyl-3-yloxymethyl]-2-hydroxymethylphenyl}methanol;
Compound 3: {4-[6-ethyl-4′-(1-ethyl-1-hydroxypropyl)-2′-propylbiphenyl-3-yloxymethyl]-2-hydroxymethylphenyl}methanol.

The compositions of Examples 2 and 3 are prepared in the following manner:

Fatty Phase A:
Weigh the constituents of the fatty phase (A): Mirasil CM5, Primol 352, Cetiol SN, Eumulgin B2, DC Emulsifier 10, butylated hydroxytoluene.

Heat to 55° C., with stirring using deflocculating device at 200 rpm.

Phase B:
Heat the PEG 400 to 55° C.
Weigh and introduce the active compound into the PEG 400, with stirring using a magnetic stirrer bar (speed 5).

Pre-Emulsification:

Slowly introduce the active phase (B) into the fatty phase (A), with stirring at 1000 rpm, and 55° C.

Cooling:

Keep stirring (800-1000 rpm) until room temperature (RT) is reached.

Phase C:

Solubilize the MgSO₄ in water, and with stirring using a magnetic stirrer bar.

Addition of Phase D:

At RT, add phase D —Ethanol Rectapur—to phase C.

Emulsification:

Slowly introduce the aqueous phase (C+D) into the pre-emulsion, with stirring at 1500 rpm.

The compositions of Examples 4 to 9 are prepared in the following manner:

Preparation of Phase A:

The hydrophobic constituents are mixed and heated to 50° C.

Preparation of Phase B1:

The active compound is solubilized in propylene glycol.

Preparation of Phase B2:

The electrolyte (MgSO₄ or NaCl) is dissolved in water.

Add Phases B2 and B3 to Phase B1 and heat to 50° C.

Phase B is incorporated into Phase A, with gentle mechanical stirring.

Example 10 is prepared in the following manner:

Preparation of Phase A:

The hydrophobic constituents are mixed and heated to 50° C.

Preparation of Phase B:

Compound 2 is solubilized in propylene glycol at 55° C.

Phase B is incorporated into Phase A, with gentle mechanical stirring at 50° C.

Preparation of Phase C:

The electrolyte is dissolved in water. Next, ethanol is incorporated.

This phase is introduced at room temperature into the emulsion, with gentle stirring.

### EXAMPLE 2

### Composition

Phase A:

| | |
|---|---|
| Mirasil CM 5 | 6.00% |
| Primol 352 | 3.00% |
| Cetiol SN | 7.00% |
| Eumulgin B2 | 1.00% |
| DC Emulsifier 10 | 3.00% |
| Butylated hydroxytoluene | 0.10% |

Phase B:

| | |
|---|---|
| PEG 400 | 58.40% |
| Compound 3 | 0.30% |

Phase C:

| | |
|---|---|
| Purified water | 14.00% |
| Magnesium sulfate 7H₂O | 1.00% |

Phase D:

| | |
|---|---|
| Ethanol Rectapur | 5.00% |

### EXAMPLE 3

### Composition

Phase A:

| | |
|---|---|
| Mirasil CM 5 | 6.00% |
| Primol 352 | 3.00% |
| Cetiol SN | 7.00% |
| Eumulgin B2 | 1.00% |
| DC Emulsifier 10 | 3.00% |
| Butylated hydroxytoluene | 0.10% |

Phase B:

| | |
|---|---|
| Propylene glycol | 58.40% |
| Compound 3 | 0.30% |

Phase C:

| | |
|---|---|
| Purified water | 14.00% |
| Magnesium sulfate 7H₂O | 1.00% |

Phase D:

| | |
|---|---|
| Ethanol Rectapur | 5.00% |

### EXAMPLE 4

### Composition

Phase A:

| | |
|---|---|
| Emulsifier 10 (lauryl methicone copolyol) | 5.00% |
| Cyclomethicone | 15.00% |
| Light paraffin oil | 15.00% |
| Cetostearyl alcohol | 3.00% |

Phase B1:

| | |
|---|---|
| Propylene glycol | 19.00% |
| Dipropylene glycol | 32.00% |
| Glycerine | 10.00% |
| Compound 3 | 1.00% |

### EXAMPLE 5

### Composition

Phase A:

| | |
|---|---|
| Emulsifier 10 (lauryl methicone copolyol) | 3.00% |
| Cyclomethicone | 10.00% |
| Paraffin oil | 10.00% |
| Ceteareth-20 | 1.00% |

Phase B1:

| | |
|---|---|
| Propylene glycol | 75.00% |
| Compound 3 | 1.00% |

### EXAMPLE 6

### Composition

Phase A:

| | |
|---|---|
| Emulsifier 10 (lauryl methicone copolyol) | 3.00% |
| Cyclomethicone | 10.00% |
| Cetearyl isononanoate | 7.00% |
| Paraffin oil | 3.00% |
| Ceteareth-20 | 1.00% |

Phase B1:

| | |
|---|---|
| Propylene glycol | 58.00% |
| Compound 2 | 2.00% |

Phase B2:

| | |
|---|---|
| Water | 10.00% |
| MgSO₄ | 1.00% |

Phase B3:

| | |
|---|---|
| Ethanol | 5.00% |

### EXAMPLE 7

### Composition

Phase A:

| | |
|---|---|
| Emulsifier 10 (lauryl methicone copolyol) | 3.00% |
| Cyclomethicone | 15.00% |
| C12-C15 alkyl benzoate | 15.00% |

Phase B1:

| | |
|---|---|
| Propylene glycol | 56.00% |
| Compound 1 | 1.00% |

Phase B2:

| | |
|---|---|
| Water | 10.00% |

### EXAMPLE 8

### Composition

Phase A:

| | |
|---|---|
| Dimethicone copolyol and cyclomethicone | 3.00% |
| Cyclomethicone | 10.00% |
| Cetearyl isononanoate | 7.00% |
| Paraffin oil | 3.00% |
| Ceteareth-20 | 1.00% |
| Zinc stearate | 1.00% |

Phase B1:

| | |
|---|---|
| Propylene glycol | 48.00% |
| Compound 3 | 1.00% |

Phase B2:

| | |
|---|---|
| Water | 20.00% |
| NaCl | 1.00% |

Phase B3:

| | |
|---|---|
| Ethanol Rectapur | 5.00% |

### EXAMPLE 9

### Composition

Phase A:

| | |
|---|---|
| Alkyl methicone copolyol | 3.00% |
| Cyclomethicone | 10.00% |
| Cetearyl isononanoate | 7.00% |
| Paraffin oil | 3.00% |
| Ceteareth-20 | 1.00% |

Phase B1:

| | |
|---|---|
| Propylene glycol | 49.30% |
| Compound 3 | 1.00% |

Phase B2:

| | |
|---|---|
| Water | 20.00% |
| MgSO₄ | 0.70% |

Phase B3:

| | |
|---|---|
| Ethanol | 5.00% |

### EXAMPLE 10

### Composition

Phase A:

| | |
|---|---|
| Alkyl methicone copolyol | 3.00% |
| Cyclomethicone | 6.00% |
| Cetearyl isononanoate | 7.00% |
| Paraffin oil | 3.00% |
| Ceteareth-20 | 1.00% |
| BHT | 0.10% |

Phase B:

| | |
|---|---|
| Propylene glycol | 58.40% |
| Compound 2 | 1.50% |

Phase C:

| | |
|---|---|
| Water | 14.00% |
| Electrolytes | 1.00% |
| Ethanol | 5.00% |

Each patent, patent application, publication and literature article/report cited or indicated herein is hereby expressly incorporated by reference.

While the invention has been described in terms of various specific and preferred embodiments, the skilled artisan will appreciate that various modifications, substitutions, omissions, and changes may be made without departing from the spirit thereof. Accordingly, it is intended that the scope of the present invention be limited solely by the scope of the following claims, including equivalents thereof.

What is claimed is:

1. A cosmetic/dermatological composition which comprises an invert emulsion containing at least one active agent sensitive to the presence of water and solubilized in a glycolic or hydroglycolic dispersed hydrophilic phase, a lipophilic continuous phase and an emulsifier having an HLB ranging from 2 to 7.

2. The cosmetic/dermatological composition as defined by claim 1, said at least one active agent sensitive to the presence of water being other than DHEA or precursor/derivative thereof and which comprises a steroid, prostaglandin, carotenoid, synthetic retinoid, derivative of vitamin D, tetracycline, minoxidil or analogue thereof.

3. The cosmetic/dermatological composition as defined by claim 2, said at least one active agent sensitive to the presence of water comprising a derivative of vitamin D.

4. The cosmetic/dermatological composition as defined by claim 3, said at least one active agent sensitive to the presence of water comprising a derivative of vitamin D₂ or D₃.

5. The cosmetic/dermatological composition as defined by claim 4, said at least one active agent sensitive to the presence of water comprising calcitriol.

6. The cosmetic/dermatological composition as defined by claim 1, said at least one active agent sensitive to the presence of water comprising (4E,6E)-7-[3-(3,4-bis-hydroxymethylbenzyloxy)phenyl]-3-ethylnona-4,6-dien-3-ol or {4-[6-ethyl-4′-(1-ethyl-1-hydroxy-propyl)-2′-propyl-biphenyl-3-yloxymethyl]-2-hydroxymethyl-phenyl}-methanol.

7. The cosmetic/dermatological composition as defined by claim 3, comprising from 0.01 to 4% by weight of said derivative of vitamin D.

8. The cosmetic/dermatological composition as defined by claim 1, comprising from 0.001 to 20% by weight of said at least one active agent sensitive to the presence of water.

9. The cosmetic/dermatological composition as defined by claim 1, said emulsifier having an HLB ranging from 2 to 7 comprising a silicone emulsifier.

10. The cosmetic/dermatological composition as defined by claim 9, said silicone emulsifier being selected from the group consisting of lauryl methicone copolyol, cetyl dimethicone copolyol, a mixture of dimethicone copolyol and cyclomethicone, and a mixture of cetyl dimethicone copolyol with polyglyceryl-4 isostearate and hexyl laurate.

11. The cosmetic/dermatological composition as defined by claim 1, further comprising a coemulsifier having an HLB of greater than 6.

12. The cosmetic/dermatological composition as defined by claim 11, said coemulsifier comprising ceteareth-20.

13. The cosmetic/dermatological composition as defined by claim 1, said dispersed hydrophilic phase comprising from 10 to 100% by volume of glycol.

14. The cosmetic/dermatological composition as defined by claim 1, said dispersed hydrophilic phase comprising at least one glycol selected from the group consisting of propylene glycol, hexylene glycol, dipropylene glycol and PEG 400.

15. The cosmetic/dermatological composition as defined by claim 1, the water activity of said dispersed hydrophilic phase being less than 0.85.

16. The cosmetic/dermatological composition as defined by claim 2, comprising:
from 0.001 to 5% of at least one active agent sensitive to the presence of water,
from 30 to 100% of glycol,
from 0.5 to 8% of an emulsifier having an HLB of from 2 to 7,
up to 5% of a coemulsifier having an HLB of greater than 6,
up to 50% of water, formulated into a topically applicable, physiologically acceptable medium therefor.

17. The cosmetic/dermatological composition as defined by claim 1, further comprising at least one cosmetic/dermatological adjuvant selected from the group consisting of hydrophilic or lipophilic gelling agents, humectants, fatty phase thickeners, preservatives, antioxidants, electrolytes, solvents, perfumes, fillers, UV-screening agents, pigments, odor absorbers, colorants and metal-chelating agents.

18. The cosmetic/dermatological composition as defined by claim 1, further comprising at least one other active agent selected from the group consisting of isoflavonoids, metalloproteinase inhibitors, carotenoids, anti-glycation compounds, NO-synthase inhibitors, vitamins, desquamating agents, compounds increasing the synthesis of glycosaminoglycans, anti-irritant compounds, compounds reducing irritation of neurogenic origin, muscle-relaxing compounds and depigmenting agents.

19. A regime or regimen for the treatment or prevention:
of dermatological conditions linked to a keratinocyte or sebocyte differentiation or proliferation disorder;
of keratinization disorders;
of dermatological conditions linked to a keratinization disorder with an inflammatory and/or immunoallergic component;
of inflammatory skin conditions not exhibiting a keratinization disorder;
of dermal or epidermal proliferation;
of dermatological disorders, bullous dermatosis and collagen diseases;
of the signs of skin aging, whether photoinduced or chronological, or for reducing actinic pigmentations and keratoses, or any skin pathologies associated with chronological or actinic aging;
of cicatrization disorders and stretch marks;
of sebaceous function disorders, acne hyperseborrhoea or ordinary seborrhoea or seborrhoeic eczema;
of dermatological conditions with an immunological component, comprising administering to an individual in need of such treatment, a thus effective amount of the cosmetic/dermatological composition as defined by claim 1.

20. The regime or regimen as defined by claim 19, comprising the treatment of acne vulgaris, comedo-type acne, polymorphic acne, acne rosacea, nodulocystic acne, acne conglobata, senile acne, secondary acne, solar acne, acne medicamentosa or occupational acne.

21. The regime or regimen as defined by claim 19, comprising the treatment of ichthyosis, ichthyosiform states, Darier's disease, keratosis palmaris and plantaris, leukoplakia, leukoplakia-like states, or skin or mucosal (buccal) lichen.

22. The regime or regimen as defined by claim 19, comprising the treatment of psoriasis, whether continuous, mucosal or ungual, arthropathic psoriasis, skin atopy, eczema, respiratory atopy or gingival hypertrophy.

23. The regime or regimen as defined by claim 19, comprising the treatment of dermal or epidermal proliferations, benign or malignant, of non-viral origin or of viral origin, verruca vulgaris, verruca plana, epidermodysplasia verruciformis, oral or florid papillomatosis, or proliferations induced by ultraviolet radiation, or baso- or spinocellular epithelioma.
